(19) United States
(12) Patent Application Publication
(10) Pub. No.: US 2018/0200455 A1
(43) Pub. Date: Jul. 19, 2018

(54) DEVICE AND METHOD FOR ENCAPSULATING A NEEDLE WITH A CORRESPONDING PLASTIC NEEDLE CAP

(71) Applicant: MEDESCIA LIMITED, London (GB)

(72) Inventors: Farhana Hassan Mohammad Kazem AHMADI, London (GB); Steven WAGSTAFF, Nottingham (GB); Neil Stephen WHITE, Nottingham (GB); Stephen Robin SMITH, Nottingham (GB); Jonathan Edward MASON, London (GB); Maximilian Vincent WOZNIAK, London (GB); Martin Georg STROHMEIER, London (GB)

(21) Appl. No.: 15/744,437

(22) PCT Filed: Jul. 15, 2016
(86) PCT No.: PCT/GB2016/052157

§ 371 Date: Jan. 12, 2018

(30) Foreign Application Priority Data
Jul. 17, 2015 (GB) 1512601.4
Dec. 17, 2015 (GB) 1522281.3

(51) Int. Cl. A61M 5/32; B09B 3/00

(52) U.S. Cl. CPC A61M 5/3278; B09B 3/005; A61M 2005/328; B09B 3/0075; A61M 2205/8206; A61M 2205/3653; A61M 2205/3606

(57) ABSTRACT

The present invention relates to an improvement to the treatment of needles for safe disposal. The device comprises a support member having at least one recess. Each said at least one recess is configured to receive a needle contained in a corresponding plastic needle cap. The device further comprises a heating element configured to heat the support member, such that the plastic material of the needle cap softens and flows to encapsulate the corresponding needle. Each said at least one recess has dimensions such that, after heating, the needle is fully encapsulated by the plastic material of the corresponding needle cap. A corresponding method is disclosed.

### CROSS-REFERENCE TO RELATED APPLICATIONS

This application is a 371 national phase U.S. non-provisional patent application, which claims the benefit of priority from Patent Cooperation Treaty Patent Application No. PCT/GB2016/052157 filed on Jul. 15, 2016, which claims the benefit of priority from GB Patent Application No. 1522281.3 filed on Dec. 17, 2015 and GB Patent Application No. 1512601.4 filed on Jul. 17, 2015, the entire disclosures of which are incorporated herein.

### FIELD

The present invention relates to a device and a method for encapsulating a needle, in particular a disposable needle that is typically used with a drug delivery pen.

### BACKGROUND

Injectable medications are commonly administered using drug delivery pens. One example is an insulin pen for injecting insulin into the body for managing Type 1 diabetes. Another example is an epi-pen used for administering adrenalin. Some pens use replaceable medication cartridges whereas others are intended to be disposable. Drug delivery pens are viewed as quick, easy and convenient alternatives to using syringes and bottled medication.

Most drug delivery pens use disposable pen needles. Pen needles comprise a short needle that is usually embedded in an annular plastic hub. The exposed end of the needle is typically sheathed with a cylindrical and tight fitting inner cap which in turn is covered by a larger plastic needle cap to protect the user and the needle.

To administer a dose of medication using such a pen, the user takes a pen needle assembly, removes the protective foil and pushes it onto the end of the pen. Next, the user twists the assembly until tight to secure the pen needle to the pen. The user then pulls off the plastic needle cap and then pulls off the inner cap to expose the needle for injection. After the medication has been injected, the needle is re-capped by the plastic needle cap (optionally preceded by first placing the inner cap back onto the needle), which is then twisted to remove the capped needle assembly from the insulin pen. The resulting assembly has the main needle part covered by the plastic needle cap but the rear part of the needle is usually exposed within the annular plastic hub. A similar procedure is followed for lancets used to test blood glucose levels, whereby after use, the exposed needle is usually inserted back into the covering cap of the lancet before disposal.

Under the safety regulations in many countries, once a needle has been used and removed, it cannot be thrown away in a normal bin, as the needle could pass on an infection if it comes into contact with others. Instead, needles must be disposed of by placing them into a sharps bin. A sharps bin is a container for holding used needles and other sharps waste until they can be disposed of safely. Once full, the sharps bin is usually taken away by a collection service or returned to a hospital, GP surgery or a pharmacy for subsequent disposal/destruction, for example, by incineration. The use of a sharps bin is not ideal on many fronts. First, it is inconvenient for the user because it requires sufficient space in the user's home, requires regular collection, and needs to be kept in a safe place out of reach of children. Second, it imposes considerable burden on healthcare providers to provide, collect and dispose of sharps bins once full. Third, many jurisdictions use methods such as incineration to dispose of sharps, which produces a wide variety of pollutants, leading to significant health and environmental hazards.

Methods and apparatuses currently exist for safely disposing of needles without the use of a sharps bin.

One method, which is disclosed in DE 20 2013 006137 U, is to destroy the needle using an electric current. The needle is used to complete a circuit between two poles whose discharge capacity is larger than the resistance of the needle. This causes the needle to heat up to a high temperature and melt. The resulting mass is sterilised and no longer sharp. However, this method requires a very high voltage and generates extremely high temperatures of 1400° C. Accordingly, this method is not suitable for home use. Besides the large amount of energy required, this method is only suitable for destroying one needle at a time and requires the needle to be precisely placed prior to destruction.

Another method is to encapsulate the needle completely. For example, U.S. Pat. No. 5,811,138 discloses a device for the encapsulation of syringes and other plastic waste having sharp elements. Syringes are placed into a chamber, which is then heated up to melt the plastic. A compaction head is used to provide a force to conform the molten plastic into a puck covering the needles. Similar devices are also disclosed in U.S. Pat. No. 5,207,994 and U.S. Pat. No. 4,860,958. One problem with these devices is that they are large devices unsuitable for home use and feature complex mechanisms with many moving parts. These complex mechanisms are necessary to cover the exposed needle part of hypodermic syringes with enough plastic to fully encapsulate them, which unlike pen needles, are not recapped after use and therefore do not have plastic directly surrounding the exposed needle. Furthermore, they require the melt chamber to be filled to a minimum level to for the apparatus to function properly, and also ensure that there is enough plastic material to encapsulate all of the syringe needles completely. These devices are also not optimal for destroying a single needle as, if a single needle were to be placed in the chamber, it could not be guaranteed that the plastic would melt so as to cover the needle.

U.S. Pat. No. 5,256,861 also discloses a device for encapsulating medical sharps. Syringes are placed into a disposable container constructed of single strength fibreboard with aluminium foil laminated to each surface. The container and syringes are placed in a chamber, which is heated up to melt the plastic syringe bodies. Upon cooling, the syringes are encapsulated in plastic and aluminium foil and can then be discarded as normal waste. The problem with this device is that it requires additional materials in the form of the disposable container to encapsulate the syringes. There is also potential for the molten plastic to leak from the aluminium foil layers.

In general, there are few, if any, devices available that are suitable for the safe disposal of pen needles. Pen needles are re-capped after use leaving the needle directly surrounded by plastic with only one exposed end, which simplifies the encapsulation process, unlike hypodermic syringes which are not re-capped after use and thus present a problem in ensuring that the needle is fully encapsulated by the plastic when the plastic is melted. Furthermore, there is demand for a device that can destroy any number of pen needles, including a small number or even just a single disposable pen needle.

The present invention aims to provide a device that is small enough for home use and that can fully encapsulate a needle contained in a corresponding plastic needle cap without the need for additional materials. The invention also aims to provide a method for encapsulating needles.

The present invention provides a device for encapsulating a needle contained in a corresponding plastic needle cap, the device optionally comprising a support member having at least one recess, each said at least one recess being configured to receive a needle contained in a corresponding plastic needle cap and optionally a heating element configured to heat the support member such that the plastic material of the needle cap softens and flows to encapsulate the corresponding needle, wherein each said at least one recess optionally has dimensions such that, after heating, the needle is fully encapsulated by the plastic material of the corresponding needle cap.

### SUMMARY

According to the invention, the support member is optionally a tray.

According to the invention, the support member typically has a plurality of recesses, optionally 2 to 100 recesses, optionally 2 to 50 recesses, preferably 3, 4, 5, 14 or 35 recesses.

According to the invention, optionally, said at least one recess has a first shape and said support member has at least one other recess of a second different shape.

According to the invention, the recesses are optionally arranged such that different shape recesses are suitable for different type needles and their corresponding plastic caps.

According to the invention, the longitudinal axes of the recesses are optionally substantially parallel to each other.

According to the invention, the recesses are optionally tessellated.

According to the invention, the recesses are optionally arranged such that adjacent recesses are orientated in opposite directions.

According to the invention, each said at least one recess is optionally configured to receive only a single needle and corresponding plastic needle cap. This allows the plastic material of each capped needle assembly to heat up and cool down more quickly so that each needle can be encapsulated and disposed of more quickly compared to processing a plurality of capped needle assemblies as one bulk mass.

According to the invention, each said at least one recess is shaped to receive a needle and corresponding plastic needle cap substantially horizontally. This helps to encourage the softened plastic material to flow around both ends of the needle.

According to the invention, each said at least one recess optionally has a length of at most 45 mm, a width of at most 30 mm and a depth of at most 20 mm.

The device of the present invention optionally further comprises a chamber containing the support member and the heating element and optionally an openable lid configured to seal the chamber when the lid is in the closed position.

According to the invention, the distance between the lid and the base of said at least one recess is optionally no greater than 28.9 mm, preferably no greater than 25 mm, more preferably no greater than 20 mm. By setting the distance between the lid and the base of said at least one recess to be shorter than the typical length of a capped needle assembly, a capped needle assembly is prevented from being placed into said at least one recess substantially vertically. Thus, a capped needle assembly is encouraged to be placed into said at least one recess in a substantially horizontal orientation, which helps to encourage plastic material to flow around both ends of the needle.

The device of the present invention optionally further comprises an inlet configured to allow air into the chamber and optionally an outlet configured to allow gases inside the chamber to leave the chamber. By allowing air to flow through the chamber, the chamber may be cooled by the air flow.

According to the invention, the inlet optionally includes a one-way valve configured to prevent gases leaving the chamber via the inlet.

According to the invention, the outlet optionally includes a filter. This allows toxic or unpleasant gaseous components given off during the heating of the plastic to be removed.

The device of the present invention optionally further comprises a temperature sensor for measuring the temperature in the chamber.

The device of the present invention optionally further comprises a locking mechanism, wherein the locking mechanism is conveniently configured to prevent the lid from being opened by a user when the measured temperature in the chamber is above a predetermined value. This helps to prevent the user from coming into contact with the hot components of the device and the hot plastic while the device is in use.

The device of the present invention optionally further comprises a pump configured to pump air through the chamber. This provides an active cooling mechanism, so that the encapsulated needles can be cooled down and disposed of more quickly.

According to the invention, the pump is optionally configured to activate after the heating element has been deactivated.

According to the invention, the pump is optionally configured to deactivate after the measured temperature in the chamber falls below a predetermined value and/or after a predetermined time period has elapsed.

According to the invention, the heating element is optionally configured to heat the support member to a temperature of from 180° C. to 330° C., optionally 180° C. to 280° C., optionally 190° C. to 250° C., optionally 200° C. to 240° C., optionally 200° C. to 230° C.

According to the invention, the heating element is optionally configured to heat the support member for a time period of from 10 minutes to two hours.

According to the invention, the lid is conveniently transparent. This allows the user to observe the progress of the encapsulation while the device is in use.

According to the invention, the support member is optionally removable from the chamber. This allows the user to remove encapsulated needle briquettes from the device and dispose of them without ever touching them.

According to the invention, the support member optionally has a rectangular shape in plan view.

According to the invention, the support member optionally has a disc shape and each said at least one recess is optionally spaced around the circumference of the disc shape. This encourages even heat distribution across each of the capped needle assemblies so that they all become encapsulated at similar rates.

According to the invention, the support member is optionally made from silicone or stainless steel.

According to the invention, the outer surface of the device has a ribbed structure.

According to the invention, the needle and plastic needle cap are typically a needle and needle cap for use with an insulin pen.

The present invention also provides a method of encapsulating a needle contained in a corresponding plastic needle cap, the method comprising optionally directly receiving into a recess of a support member a needle contained in a corresponding plastic needle cap, and optionally heating the support member such that the plastic material of the needle cap softens and flows to fully encapsulate the corresponding needle.

According to the invention, plural needles and corresponding plastic needle caps are optionally received into respective plural recesses of the support member.

According to the invention, the method further comprises a step of cooling the support member so that the softened plastic hardens whilst encapsulating the corresponding needle.

According to the invention, the cooling optionally comprises active cooling created by pumping air through the chamber.

The method of the present invention optionally further comprises removing the support member and disposing of the encapsulated needle(s).

The method of the present invention optionally further comprises locking the lid while heating and cooling.

The method of the present invention optionally further comprises preventing the needle contained in its corresponding plastic needle cap from being received into a recess with the needle in the generally vertical direction.

The method of the present invention optionally further comprises filtering air in the chamber to remove toxic or unpleasant gaseous components.

The method of the present invention optionally further comprises measuring the temperature of the air in the chamber and optionally unlocking the lid when the temperature falls below a predetermined value.

According to the invention, the predetermined value is optionally in the range 30° C. to 70° C., optionally 40° C. to 60° C., optionally 40° C. to 50° C.

According to the invention, the support member is optionally heated to a temperature of from 180° C. to 330° C., optionally 180° C. to 280° C., optionally 190° C. to 250° C., optionally 200° C. to 240° C., optionally 200° C. to 230° C.

The present invention also provides a support member for a needle encapsulating device, said support member comprising one or more recesses for supporting a capped needle assembly.

### BRIEF DESCRIPTION OF THE DRAWING FIGURES

The present invention will now be described, by way of non-limitative example only, with reference to the accompanying drawings, in which:

FIG. 1 is an exploded side view of a capped pen needle assembly.

FIG. 2 is a perspective view of a first embodiment of a device according to the invention.

FIG. 3 is an exploded perspective view of the device shown in FIG. 2.

FIG. 4 is a plan view of the support member of the device shown in FIG. 2.

FIG. 5 is a perspective view of the support member of the device shown in FIG. 2.

FIG. 6 is a photograph showing a support member with some capped needle assemblies placed individually within recesses of the support member.

FIG. 7 is a perspective view of a second embodiment of a device according to the invention.

FIG. 8 is an exploded perspective view of the device shown in FIG. 7.

FIG. 9 is a plan view of the support member of the device shown in FIG. 7.

FIG. 10 is a perspective view of the support member of the device shown in FIG. 7.

FIG. 11 is a perspective view of a support member according to the invention.

FIG. 12 is a perspective view of a support member according to the invention.

### DESCRIPTION OF THE PREFERRED EMBODIMENT

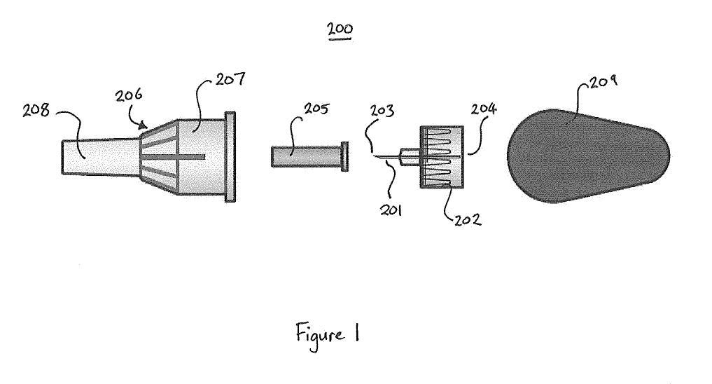

FIG. 1 shows a capped needle assembly 200 that would typically be used to inject medication with a drug delivery pen.

The capped needle assembly 200 for use with a drug delivery pen comprises a needle 201 embedded in an annular plastic hub 202 such that one end 203 of the needle protrudes from the plastic hub 202. While the other end 204 of the needle does not protrude from the annular cap in the longitudinal direction, it is nevertheless exposed as the rear end of the annular cap is open to allow engagement with the drug delivery pen. An inner cap 205 is provided to sheath the protruding end of the needle 201. The inner cap 205 is of a cylindrical shape and snuggly fits around the needle 201. The assembly includes a corresponding plastic cap 206, which is configured to contain and surround the needle 201, plastic hub 202 and inner cap 205. The plastic cap 206 has a generally tapered shape and comprises a large end 207 and a small end 208. The large end 207 houses and surrounds the plastic hub 202 and the small end 208 houses and surrounds the inner cap 205, which in turn surrounds the needle 201. The capped needle assembly 200 is generally provided in the assembled configuration, namely with the inner cap 205 snuggly fitted on to the needle 201 and with the plastic cap 206 engaged over and around the inner cap 205 and the plastic hub 202. The exposed rear end of the needle 204 is protected by a paper or foil peel-off tab 209 that is attached to the opening at the large end 207 of the plastic cap 206. After use, the capped needle assembly 200 comprises the same components arranged in the same way, but may exclude the inner cap 203. Each needle 201 generally has a corresponding plastic cap 206, that is to say there will be one plastic cap 206 for each needle 201. The corresponding plastic cap 206 generally covers and contains the corresponding needle 201. Although not clearly shown in FIG. 1, the rear end of the needle 204 is somewhat exposed after use and presents a needle stick hazard. This is the reason procedures are in place for the proper disposal of these pen needles.

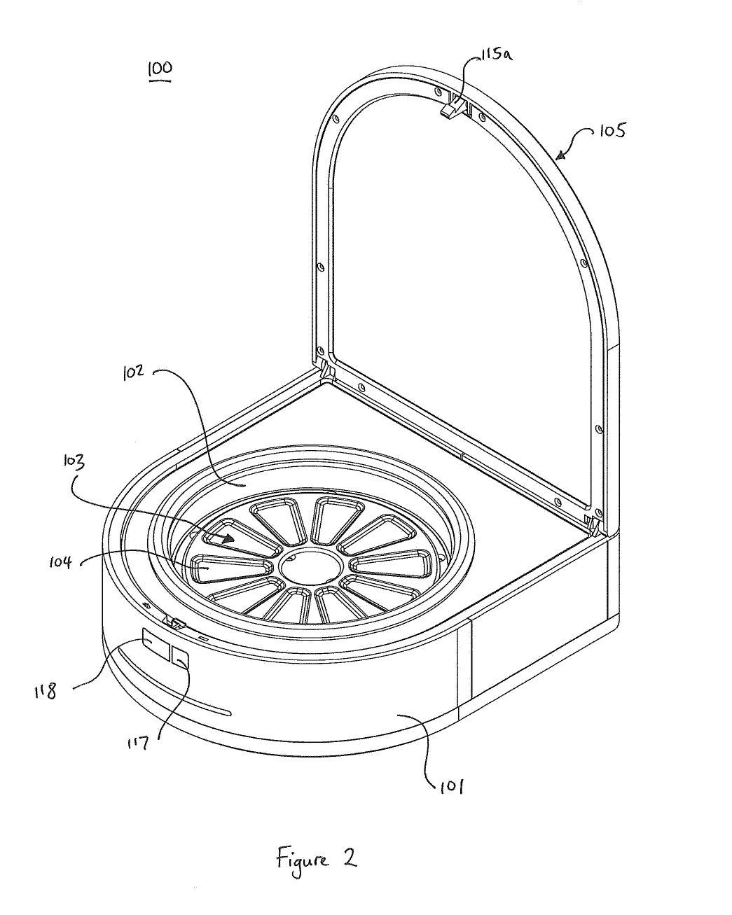

An embodiment of a device for encapsulating needles is shown in FIG. 2. The device 100 of FIG. 2 comprises a base 101 having a chamber 102. Located within the chamber 102 is a support member 103 comprising at least one recess 104, which will be described in further detail later. The device 100 further comprises a lid 105 configured to close over the base 101 and seal the chamber 102.

The general concept of operation is that the user places a used pen needle 201 (complete with its corresponding plastic cap 206) into a recess 104 of the support member 103. As many or as few pen needles as are required may be placed in the device 100, up to a maximum limit given by the total number of recesses 104. Then, the lid 105 is closed to seal the chamber 102 and heat is applied. This heat serves to soften the plastic of the plastic cap 206 (and also the plastic hub 202 and inner cap 205 if present) so that it flows to encapsulate the needle 201 forming a small brick of plastic, termed a briquette here. The lid 105 preferably remains locked during this heating phase. Once the needle 201 has been encapsulated by plastic, the encapsulated needle briquettes are allowed to cool, or are actively cooled. The optional lock may then be released and the support member 103 may be removed to allow ready disposal of the briquettes in a standard waste dustbin.

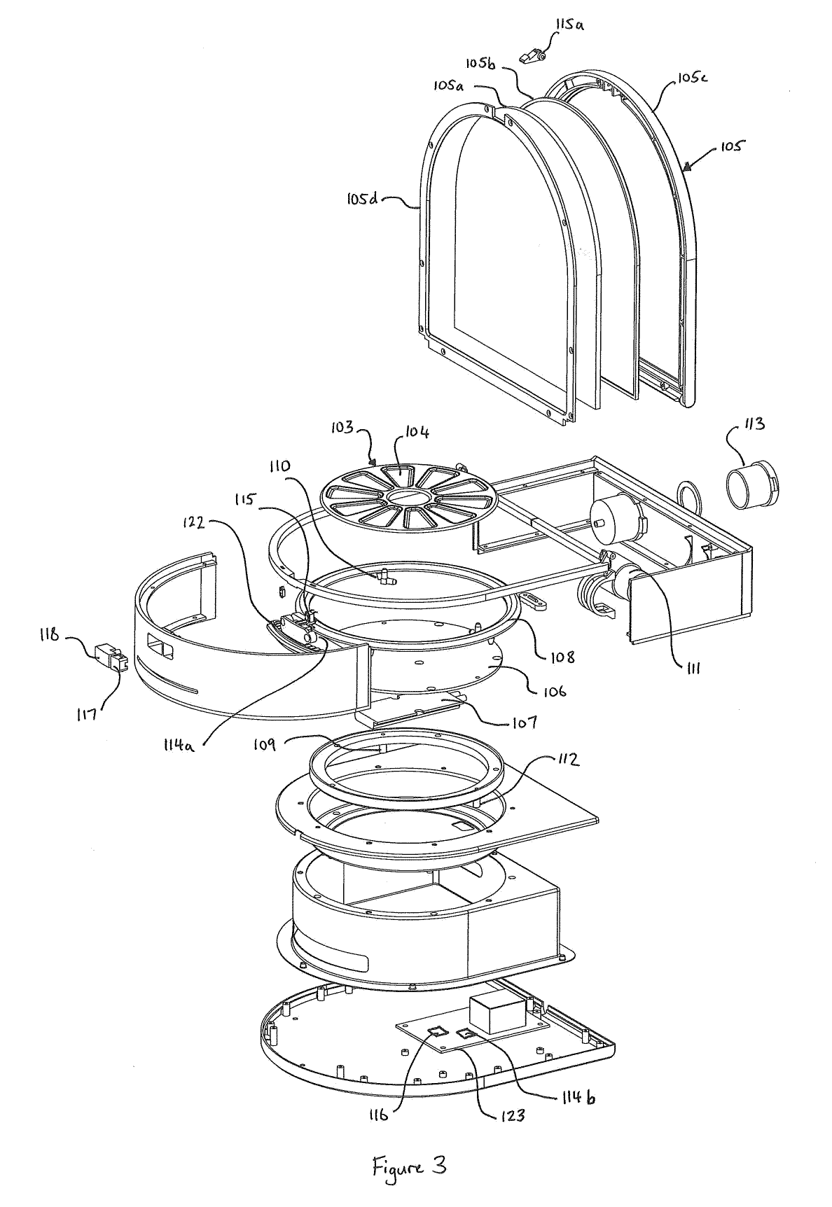

FIG. 3 shows the interior components of device 100. In addition to the support member 103, an optional heating plate 106 and a heating element 107 are provided in the chamber 101. Around an outer edge of the chamber 102 is an optional gasket 108, which is in contact with the lid 105 when the lid 105 is in a closed position.

The lid 105 is optionally made from a transparent material or materials so that the user can see inside the chamber 102 when the lid 105 is closed.

The lid 105 of the device 100 may comprise a plurality of layers 105a, 105b sandwiched between a lid cover 105c and lid base 105d. For example, the lid 105 may comprise an inner layer of heat resistant glass 105a and an outer layer of impact resistant glass or plastic 105c with an optional air or vacuum gap between them. This helps to insulate the chamber 102 to prevent the outside of the device from becoming too hot to touch when in use.

The device 100 shown in FIGS. 2 and 3 further comprises an inlet 109 configured to allow air to flow into the chamber. The inlet 109 may have a one-way valve 110 configured to allow air into the chamber 102 but to prevent gases from leaving the chamber 102 via the inlet 109.

During the heating and encapsulating process, gases may be given off which are toxic and/or unpleasant. The device 100 thus further comprises an outlet 112 configured to allow gases inside the chamber 102 to leave the chamber 102. The outlet 112 may have a filter 113 to filter out toxic fumes, for example. The filter is preferably an activated carbon filter as is known in the art.

The device 100 may further comprise a pump 111, preferably configured to pump air out of the chamber 102. In the drawings, the pump is disposed to the rear of the chamber and once activated, creates negative pressure in the chamber 102. This causes air to be drawn into the chamber 102 through the inlet 109. This air will pass over the support member 103, recesses 104 and any encapsulated pen needles, as well as the heating plate 106 and heating element 107. The air will serve to cool the components that it comes into contact with. This active cooling shortens the time between the needle encapsulation being complete and the encapsulated needles being cool enough to be touched by the user.

A temperature sensor 114 mounted on a PCB 122, 123, for example, may be provided to measure the temperature inside the chamber. The temperature sensor 114 may measure the temperature of the air in the chamber and/or may measure the temperature of a component in the chamber, such as the heating plate 106, the heating element 107 or the support member 103. Preferably, two thermal sensors 114a, 114b are used, one measuring the temperature of a component in the chamber and the other measuring the temperature of the air in the chamber. The temperature sensor 114a for measuring a component in the chamber may be mounted on PCB 122 and can be used to ensure that the correct temperature has been reached during the heating cycle. The temperature sensor 114b for measuring the temperature of the air in the chamber may be mounted on PCB 123 and can be used to ensure that the general temperature in the chamber is cool enough to signal the end of the cooling cycle.

The lid 105 and chamber 102 may be provided with a locking mechanism 115 to prevent the lid 105 from being opened by a user.

The device 100 may further comprise a processor 116 configured to control and/or receive signals from various elements of the device 100, such as the heating element 107, the pump 111, the temperature sensor 114 and the locking mechanism 115. The processor 116 is preferably pre-programmed to perform all of the steps necessary to achieve a complete encapsulation. Thus, once the lid has been closed and the start button has been pressed, the processor 116 will receive a signal from the locking sensor to indicate that the lid is properly closed and may start an optional countdown waiting period. This period may be in the range of 10 seconds to 5 minutes, and preferably is around 30 seconds. This waiting period gives the user the opportunity to open the lid and insert further capped needle assembly 200 for encapsulation. Following this waiting period, or immediately after the start button has been pressed if no waiting period is implemented, the processor 116 will send a signal to the locking mechanism 115 to cause the lid to be locked, so that it may not be opened by the user. Then, the processor 116 is programmed to activate the heating element 107 so as to bring the temperature in the chamber to an appropriate level for softening the plastic and encapsulating the needle. This temperature is preferably in the range of 180° C. to 330° C., preferably 180° C. to 280° C., more preferably 190° C. to 250° C., more preferably 200° C. to 240° C., more preferably 200° C. to 230° C. Optionally, a temperature sensor 114a that measures the temperature of a component in the chamber, such as the heating plate 106, can be used to feed back to the processor that the correct temperature has been reached. Optionally, closed loop control can be applied to keep the temperature in the chamber at the desired level. The processor 116 is arranged to maintain the temperature in the chamber at the elevated level for a predetermined period of time, for example 10 minutes to 1 hour, preferably 20 minutes to 45 minutes, more preferably 25 minutes to 40 minutes, most preferably around 35 minutes. The temperature and period of time are selected to ensure that the plastic softens and flows so that the needle 201 is fully encapsulated. The processor 116 also operates various optional LED indicators that are visible to the user. An LED indicator can indicate when heating is taking place, when cooling is taking place and when the lid is locked or unlocked. Once the heating cycle has been completed, the processor 116 activates the cooling cycle. This can be a passive cooling cycle whereby the heating element 107 is deactivated and the temperature in the chamber is allowed to equalise with the surrounding chamber naturally. The temperature sensor 114b may be used to monitor the temperature in the chamber during the cooling cycle. Optionally, active cooling can be implemented whereby a pump 111 is used to circulate air through the chamber. Preferably, the pump 111 sucks air out of the chamber to create a negative pressure but equally the pump could be used to force air into the chamber to create positive pressure. The pump 111 is activated and deactivated by the processor 116. Once the cooling cycle is finished, which may be signalled either by the temperature in the chamber or the temperature of a component in the chamber falling below a predetermined value, e.g. between 35-45° C., preferably 40° C., or by the elapsing of a predetermined amount of time, for example 20 to 50 minutes, or by some combination of these criteria (for example the cooling cycle may be deemed complete either once 30 minutes have elapsed or once the temperature falls below 40° C.), the lid can be unlocked. Possibility, the processor 116 may be configured to merely deactivate the pump following the elapsing of a pre-set time and may be configured to unlock the locking mechanism 115 once the temperature in the chamber falls below a predetermined value. Alternatively, the pump may be deactivated and the locking mechanism 115 may be unlocked substantially simultaneously by the processor 116 upon the elapsing of a certain period of time, upon the temperature falling below a predetermined temperature or upon the reaching of one or both of these criteria as explained above.

The heating and cooling cycles are each generally timed to take between 20 minutes and 1 hour, preferably between 30 minutes and 50 minutes, more preferably between 35 minutes and 40 minutes. The preferred value for the heating cycle is 40 minutes and for the cooling cycle is 30 minutes. The heating cycle does not have to be longer than the cooling cycle and may be shorter than the cooling cycle.

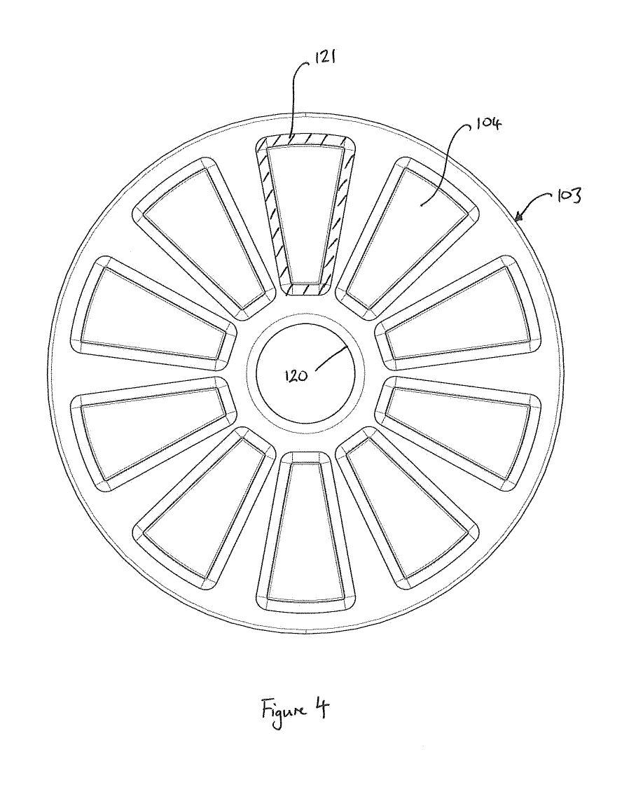

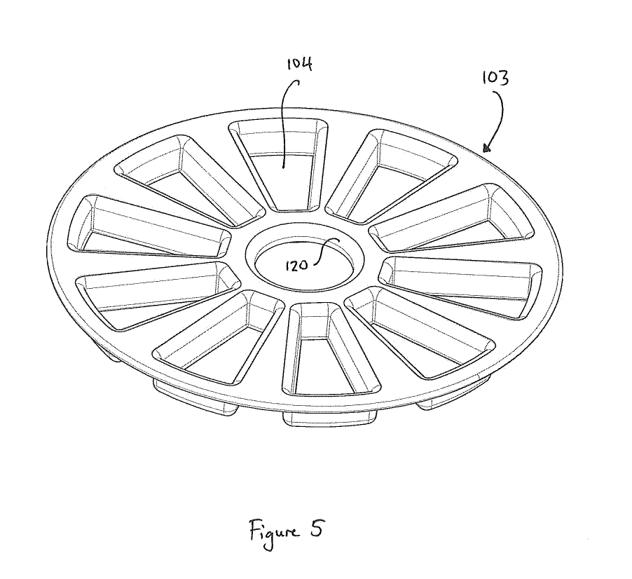

FIGS. 4 and 5 show the support member 103 comprising at least one recess 104. Ten recesses 104 are shown in the Figures, but the support member 103 is not limited to this number of recesses and may comprise, for example, one to twenty-five recesses, preferably five to twenty recesses, more preferably seven to twelve recesses, depending on the size of the support member 103. The support member 103 preferably has a disc shape, but could have other shapes such as a rectangular shape. In the case of a disc-shaped support member 103, the recesses 104 are preferably spaced around the circumference of the support member 103 as shown in FIG. 4.

The support member 103 may be integrally formed with the device 100 or may be removable from the chamber 102. The support member 103 may take the form of a tray as shown in the Figures. The support member 103 may be provided with a hole and/or indentation/protrusion to assist with manual removal of the support member 103 from the chamber 102.

Operation of the device 100 will now be described. First, the lid 105 is opened—preferably by the user. This may be done with the use of an optional opening button 118. Pressing the opening button 118 releases a catch 115a from the locking mechanism 115 (see FIG. 3).

One or more capped needle assemblies 200 are placed into respective recesses 104 of the support member 105. In general, each recess 104 is sized and dimensioned so as to accommodate a single capped pen needle. Not all recesses 104 are required to be occupied in order to use the device 100.

As shown in FIG. 1, the base 207 of the capped needle assembly 200 is wider than its tip 208 and therefore the amount of plastic material varies along the length of the capped needle assembly 200. To take this into account, each recess 104 optionally has a shape that approximately follows the outer shape of a capped needle assembly 200 (i.e. in a plan view, each recess 104 has two opposite sides that converge together and two opposite sides that are generally parallel, with one of these two opposite parallel sides being larger than the other. This results in a generally trapezoid shape for the recess as shown in the Figures. This trapezoid shape itself facilitates the arrangement of multiple recesses around the circumference of the support member 103. As shown in the embodiment, the shape of each recess is preferably an isosceles trapezoid, i.e. symmetrical about a generally longitudinal axis, where the longitudinal direction is the direction that the needle points when placed into the recess. When a capped needle assembly 200 is placed into its corresponding recess, it generally points inwardly and, in the embodiment shown, each capped needle assembly 200 points to the centre of the generally circular support member 103.

According to this embodiment of the invention, the recesses 104 are generally regularly disposed over the support member 103 and are separate from one another. This serves to ensure that each needle 201 is separately and individually encapsulated by its corresponding plastic cap 206. There is no overflow of plastic from adjacent or other recesses 104.

Once the support member 103 has been loaded, the lid 105 is closed and the start button 117 is depressed by the user. Following an optional waiting period, the processor 116 causes the locking mechanism 115 to engage the lock, thereby preventing the lid 105 from being opened. The heating element 107 is then activated and rapidly reaches a predetermined temperature. Optionally, a heating plate 106 may be used to encourage an even distribution of heat underneath the support member 103. The support member 103 is heated such that the plastic material of each capped needle assembly 200 (i.e. the plastic material of at least the plastic needle cap 204 and optionally the plastic hub 202 and the inner cap 205, if present) becomes soft and flows to encapsulate the corresponding needle 201.

Encapsulation of each needle 201 is conveniently achieved by each recess 104 having dimensions such that, after heating, each needle 201 is fully encapsulated by the plastic material of its corresponding plastic cap 206.

If a recess 104 were to be too long and/or too wide, the softened plastic material may not be of a sufficient depth to fully cover the needle 201. If each recess 104 is too shallow, then the softened plastic material may flow out of the recess 104. Preferably, each recess 104 has an axial length of at most 45 mm, an average width of at most 30 mm and a depth of at least 1.0 mm and at most 20 mm. The average width here is the width at the centre of the recess but this may also be defined as the mean average of the lengths of the two parallel sides of the recess. Preferably, each recess 104 has an axial length of at most 40 mm, preferably at most 38 mm, more preferably at most 35 mm. So as to accommodate most typical pen needle assemblies, the axial length should be greater than 25 mm, preferably greater than 27 mm or preferably greater than 29 mm, most preferably greater than 32 mm. Preferably, each recess 104 will have an average width of at most 25 mm, more preferably at most 20 mm, most preferably at most 15 mm. The small end of the recess 104 where the small end 208 of the plastic cap 206 sits preferably has a width in the range 6 mm to 10 mm, more preferably 6.5 mm to 9 mm, more preferably still 7 mm to 8 mm. The most preferred width of the small end is 7.5 mm. The large end of the recess 104 where the large end 208 of the plastic needle cap 206 sits preferably is 15 mm to 25 mm, more preferably 17 mm to 22 mm, most preferably 18 mm to 20 mm. The most preferred dimension for the large end of the recess is 19 mm. The axial length of each recess 104 is preferably no more than twice the maximum width of the recess 104, or no more than 2.5 times the maximum width of the recess 104, or no more than three times the maximum width of the recess 104. Each recess 104 preferably has a depth of at least 1.0 mm, more preferably at least 3.0 mm, most preferably at least 5.0 mm. The depth is preferably no more than 20 mm, more preferably no more than 15 mm, most preferably no more than 10 mm.

As noted above, the support member 103 is heated, directly or indirectly, by the heating element 107. The processor 116 may be configured to activate the heating element 107 once the device 100 is switched on. Optionally, the lid 105 may be required to be in the closed position before the heating element 107 can be activated.

Once the plastic material has cooled down sufficiently, the encapsulated needle(s) 201 in the form of briquettes may be removed from the device 100 for disposal.

As noted above, each recess 104 is preferably configured to receive only a single capped needle assembly 200. This allows the plastic material of each capped needle assembly 200 to heat up and cool down more quickly so that each needle 201 can be encapsulated and disposed of more quickly compared to processing a plurality of capped needle assemblies 200 as one bulk mass.

To assist with full encapsulation of the needle 201, each recess 104 is preferably shaped to receive a capped needle assembly 200 substantially horizontally (i.e. the capped needle assembly 200 can be placed into a recess 104 such that the axis of the capped needle assembly 200 lies substantially parallel to the base of the recess 104). This horizontal orientation of the capped needle assembly 200 helps to encourage the softened plastic material to flow around both ends of the needle 201. If the capped needle assembly 200 were to be orientated vertically, there is a possibility that one end of the needle 201 would be protruding from the plastic material once hardened. To help ameliorate this, the invention includes an optional means for preventing the heating of a substantially vertical needle.

In particular, to help ensure that the capped needle assemblies 200 are placed in the recesses 104 only substantially horizontally (i.e. not substantially vertically), the distance (i.e. the straight-line distance) between the base of each recess 104 and the lid 105 of the device 100 may be set to be shorter than the length of the capped needle assembly 200. One typical capped needle assembly has a length of 29 mm and so, for example, the distance between the base of each recess 104 and the lid 105 may be set to no greater than 28.9 mm, optionally no greater than 27 mm, optionally no greater than 25 mm. Another typical capped needle assembly has a length of 27 mm and so the distance between the base of each recess 104 and the lid 105 may be set to be no greater than 26.9 mm, optionally no greater than 26 mm, optionally no greater than 24 mm. To enable the device to be used optimally with a variety of different capped needle assemblies from different manufacturers, a distance between the base of each recess 104 and the lid 105 may be set such that it is shorter than the length of all, or substantially all, typical pen needle assembles. For example, the distance could be set so that it is no more than 20 mm, preferably no more than 18 mm, preferably no more than 17 mm. Naturally, the distance should be sufficient to allow the pen needle assembly to rest in the recess in a substantially horizontal configuration. The typical diameter of the large end 207 of the plastic cap 206 is 15 mm or 16 mm. Thus, the distance between the base of each recess 104 and the lid 105 will preferably be greater than 15 mm, more preferably greater than 16 mm. To enable some air to circulate around the pen needle assembly 200 during the cooling procedure, the distance between the base of each recess 104 and the lid 105 may be set to be greater than 18 mm. A distance of about 20 mm works well in practice. In this way, the lid 105 is unable to close when a capped needle assembly 200 is orientated vertically in a recess 104 but is able to close if a capped needle assembly 200 is not orientated vertically.

The support member 103 is preferably in the form of a tray. The support member may have a hole 120 and this hole is preferably arranged in the centre of the support member 103. The hole 120 facilitates the user removing the support member 103 by placing a finger through the hole. This enables the user to remove all of the encapsulated briquettes at once and dispose of them in the bin in a single action. The hole 120 in the support member 103 is preferably sized so as to allow a user's finger to pass through it. It is preferably sized with a diameter or largest dimension in the range of 1 cm to 6 cm, preferably 2 cm to 4 cm, most preferably 2 cm to 3 cm, ideally around 2.5 cm.

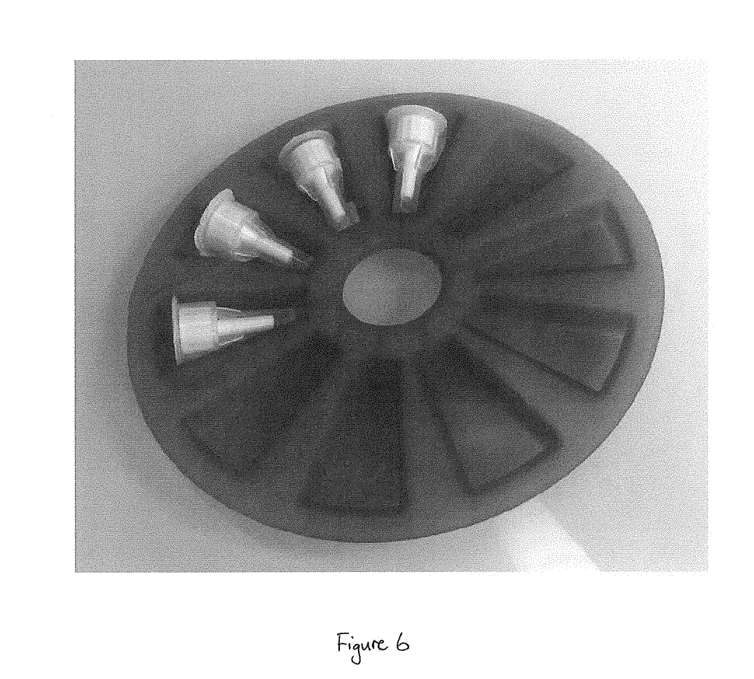

FIG. 6 shows a support member in the form of a silicone tray with four pen needle assemblies 200 inserted in respective recesses 104. It can be seen from this Figure that the recesses 104 are appropriately shaped and sized to receive a capped pen needle assembly and to keep it separate from other capped pen needle assemblies 200 on the support member 103. As a result, each needle 201 is encapsulated only by the plastic in its own corresponding plastic needle cap 206. An indentation and/or protrusion may be provided instead of, or in addition to, a hole.

Each recess 104 is preferably bevelled rather than sharp-edged. The bevel preferably spans a distance of around 2.0 mm when viewed in plan view, as shown in FIG. 4. In FIG. 4, the bevel 121 of one of the recesses is identified using cross-hatching. The radius of the bevel is about 1.0 mm.

The tray is ideally made from silicone material as this material is able to withstand heat and distribute it appropriately to the various pen needle assemblies 200. Furthermore, the material is soft without presenting any sharp edges. The material is also cost effective and may readily be molded to the desired shape. Furthermore, the user is readily able to “pop” each melted needle briquette out of the silicone recess 104 by bending the silicone material. Thus, with the present invention, the user is able to remove the briquettes from the device and readily place them into the bin without ever touching them.

The support member 103 may alternatively be made from stainless steel or similar other non-corrodible material. The silicone material however has the benefit of cooling down more quickly than stainless steel.

The support member 103 is preferably of single-piece construction.

The present invention has numerous benefits compared to the prior art. The various components of the device 100 are simple and do not require high tolerance machining. Aside from the lid 105, there are no moving parts and no plungers or compaction mechanisms are required. The device 100 is able to encapsulate even a very small number of capped needle assemblies 200, for example one, and equally is able to encapsulate a larger number, for example 10. The overall design is compact and thus suitable for home use. The whole procedure from placing the used capped pen needle assembly 200 into the device 100 to removing the encapsulated briquette takes around an hour. This compares to at least 2-3 hours for prior art devices which generally require complete melting of the plastic such that the plastic flows to the bottom of the chamber with all of the metal components.

The device is safe for home use because the air filter 113 and air pump 111 ensure that no toxic or unpleasant fumes can escape. Further, the double-glazed lid design ensures that the external surface of the device 100 remains cool even while high temperatures are developed inside of the device 100.

The support member design means that the briquettes can be disposed of without needing to touch them. In particular, the removable nature of the support member helps to achieve this.

The device does not require any consumable items, such as disposable chambers or the like, and uses only the plastic already part of the capped pen needle assembly 200 to achieve encapsulation. The support member 103 itself can be re-used many times.

The device is user friendly and intuitive to operate. Once activated, the procedure is run by the device itself without any need for user programming. The transparent lid 105 allows the user to observe the operation and optional LED status indicators may be used to provide feedback on the stage of the procedure.

The device 100 is easy to clean as there is only a single chamber 102 with a single support member 103 located in it. The removable nature of the support member 103 means that it can be readily cleaned if required.

The process is repeatable thereby ensuring that needles 201 are always fully encapsulated by plastic.

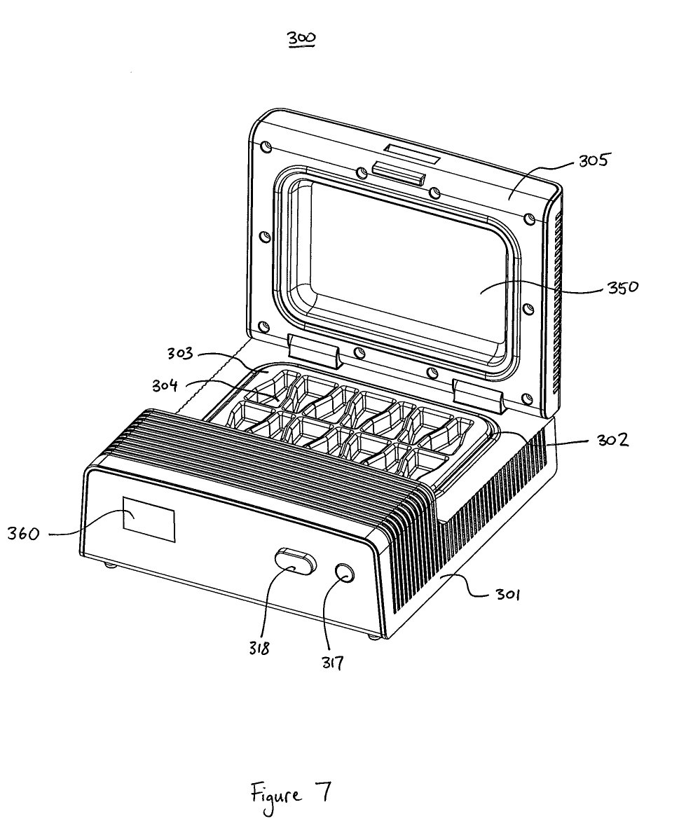

A second embodiment of a device 300 for encapsulating needles is shown in FIGS. 7-10. Similar to the first embodiment, the second embodiment typically comprises a base 301 having a chamber 302. Located within the chamber 302 is a support member 303 comprising at least one recess 304. The device 300 further comprises a lid 305 configured to close over the base 301 and seal the chamber 302.

The outer surface of the device 300 is optionally made from plastic, e.g. ABS plastic. The device 300 preferably has a ribbed structure over at least a portion of the outer surface. The ribbed structure typically comprises alternating peaks and valleys, which increases the outer surface area of the device 300 compared to a flat surface and provides increased heat transfer away from the device. As a result, the device 300 is cool enough to touch at all times, even during the heating process.

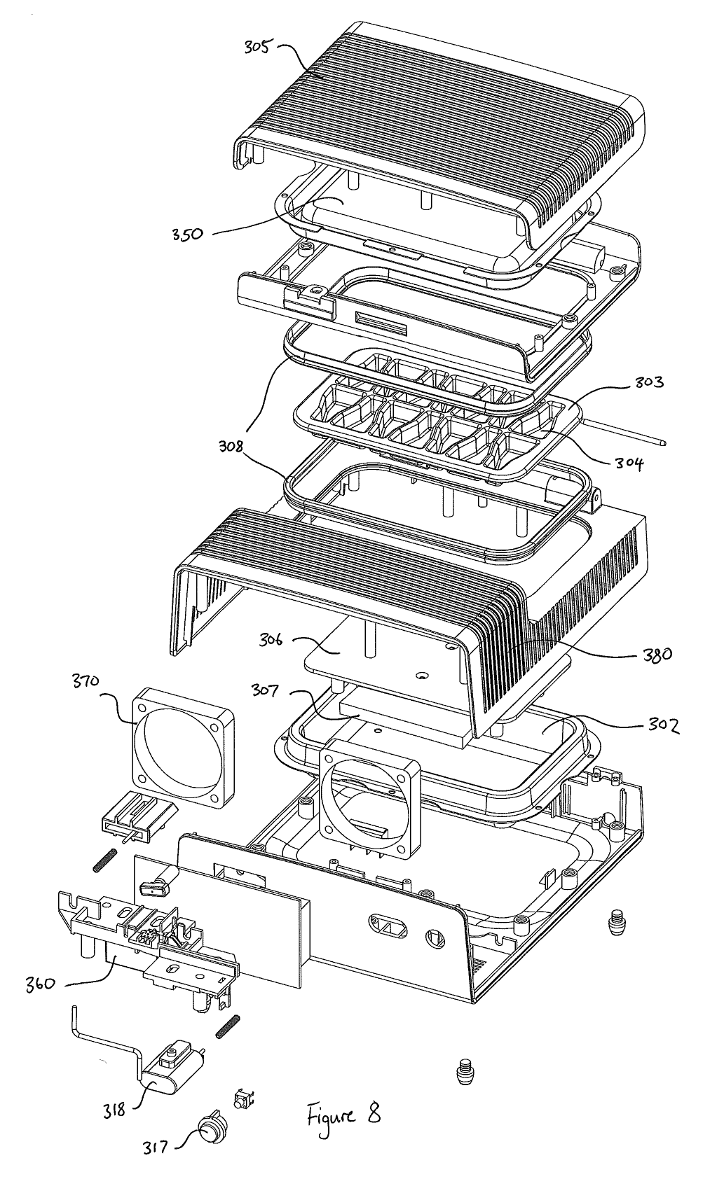

FIG. 8 shows preferable internal components of the device 300. The chamber 302 preferably comprises a heating plate 306 and heating element 307. The heating plate 306 is preferably made from aluminium for effective heat transfer from the heating element 307 to the chamber 302. The heating surface of the heating element 307 is preferably the same area as the heating plate 306 and is optionally positioned directly below the heating plate 306, so that the chamber 302 is heated evenly with no hotspots. The walls of the chamber 302 are preferably made from stainless steel, which has a relatively low thermal conductivity and thus helps to retain heat within the chamber 302 and minimise heat transfer to the outer surface of the device 300. The chamber 302 may also be surrounded with an insulating material, preferably a mineral wool (e.g. ROCKWOOL®), to help with heat retention and to minimise heat transfer to the outer surface of the device 300 so that the outer surface is cool enough for the user to touch while the device is in operation.

On the underside of the lid 305, a recess may be provided in which a chamber cover 350 sits. The chamber cover 350 is preferably dish-shaped and at least the same length and width as the chamber 302, such that when the lid is in a closed position, the chamber 302 and the chamber cover 350 form an enclosed box around the support member 303. The chamber cover 350 is preferably made of the same material as the chamber 302 (e.g. stainless steel) and is optionally surrounded with an insulating material, preferably a mineral wool (e.g. ROCKWOOL®) to help with heat retention and to minimise heat transfer to the outer surface of the lid 305 so that the lid is cool enough for the user to touch when the device is in operation.

The chamber 302 and chamber cover 350 provide a barrier between the hot temperatures inside the device 300 and the outer surface of the device 300 during operation. This barrier may be further improved by surrounding the chamber with an insulating material. Due to this barrier, the outer surface of the device 300 remains cool enough (at approximately 40° C.) for the user to touch during operation, while minimising the thickness of the outer surface material, which minimises the overall size of the device 300. In particular, the device 300 can provide a safe outer surface temperature using an outer surface material thickness of approximately 10 mm.

The dish shape of the chamber cover 350 also provides a space in the lid 305 in which the portions of plastic needle caps 206 that stand proud from the upper surface of the support member 303 may occupy when the lid 305 is fully closed. This allows the support member 303 to be positioned closer to the upper surface of the base 301, or stand proud of the upper surface of the base 301, thereby facilitating insertion and removal of the support member 303 from the device 300.

Around an outer edge of the chamber 302 is an optional gasket 308, which is in contact with the lid 305 when the lid is in a closed position and which helps to provide a tight seal around the chamber 302. A gasket 308 could alternatively or additionally be provided on the lid 305, so that the gasket 308 makes contact with the outer edge of the chamber 302 when the lid 305 is in a closed position. The support member 303 is optionally sized such that the rim of the support member 303 is sandwiched between the base 301 and the lid 305, thereby acting as a gasket to seal the chamber 302.

The front of the device 300 typically comprises a start button 317 and a lid opening button 318, which operate as described for the first embodiment. The locking mechanism of the present embodiment can be the same as that described for the first embodiment. The front of the device 300 preferably further comprises an LED or LCD display 360, which is conveniently configured to display the time remaining during each stage of the encapsulation process. The display 360 may also show status messages, indicating, for example, when the encapsulation process has finished. The display 360 may also indicate errors, e.g. in the case where the user presses the start button 317 to start the encapsulation process when the lid 305 is open. Alternatively or in addition to the display 360, the device 300 may comprise an LED indicator similar to the first embodiment. The device 300 may also comprise a speaker or buzzer (not shown) configured to provide an audible alert when the encapsulation process has started and/or finished, or when there is an error.

The base 301 of the device 300 preferably comprises a hollow section for housing electric components and an air ventilation system, which has the same components and functions as previously described for the first embodiment. The outer surface of the device 300 surrounding the hollow section may include at least a portion comprising openings (vents) 380 to assist with ventilation. At least one fan 370 may also be provided within the hollow section to suck in air from outside the device 300 through the openings 380 to assist with cooling. This allows the heating element 307 to be heated to higher temperatures while maintaining the electrical components at a cool enough temperature, so that the heating process can be carried out in less time compared to a device that does not have fan-assisted cooling.

The heating temperatures, heating and cooling times and control of the encapsulation process of the present embodiment are similar to those of the first embodiment. Preferably, a heating and cooling cycle takes approximately 70 minutes in total, with a heating time of approximately 40 minutes and a cooling time of approximately 30 minutes. The cooling time may be based on a predetermined elapsed period of time, or when the temperature of a component or the air in the chamber 302 falls below a predetermined value, such as 40° C.

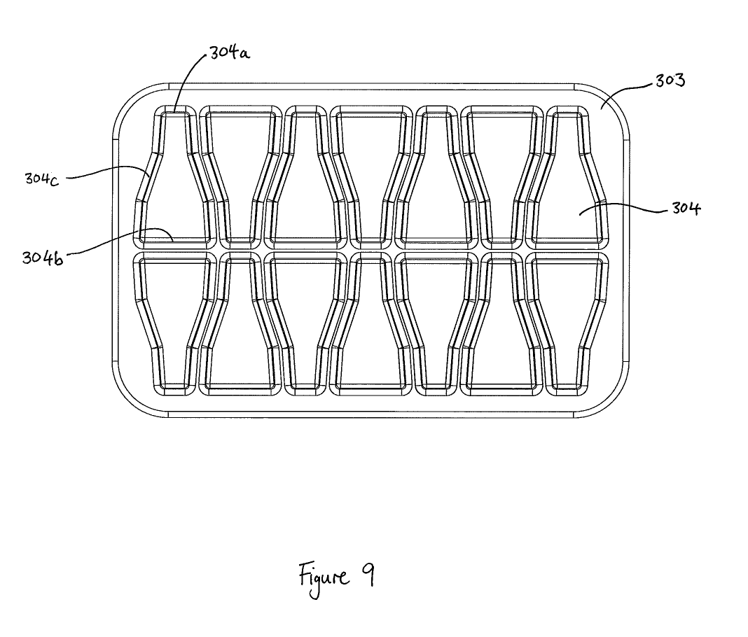

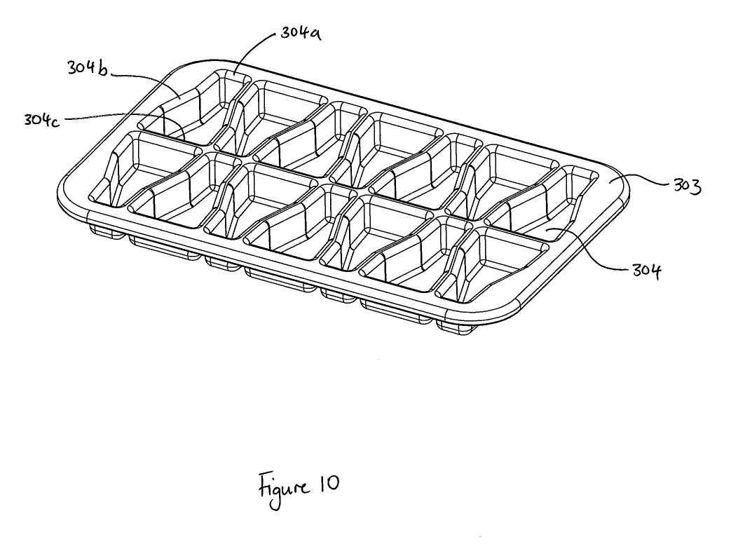

FIGS. 9 and 10 show a support member 303 that may be used with the device 300 according to the present embodiment. The support member 303 preferably has a rectangular shape in plan view, but could have other shapes such as a square or any N-sided polygon, where N>3. The options for the material of the support member 303 are the same as for the support member 103 of the first embodiment.

FIG. 9 shows the shape of a typical recess 304 in plan view. The shape of the recesses 304 preferably generally follows the shape of a capped needle assembly 200. The recesses 304 preferably have a generally tapered shape and comprise a large end 304a and a small end 304b with an optional tapered section 304c in between the large end 304a and the small end 304b. Each recess may be symmetrical about a longitudinal axis passing through the large end 304a and the small end 304b. The recesses 304 are not limited to the shape shown in FIG. 9 and may instead have an isosceles trapezoid shape similar to the recesses 104 described in relation to the first embodiment and shown in FIG. 4.

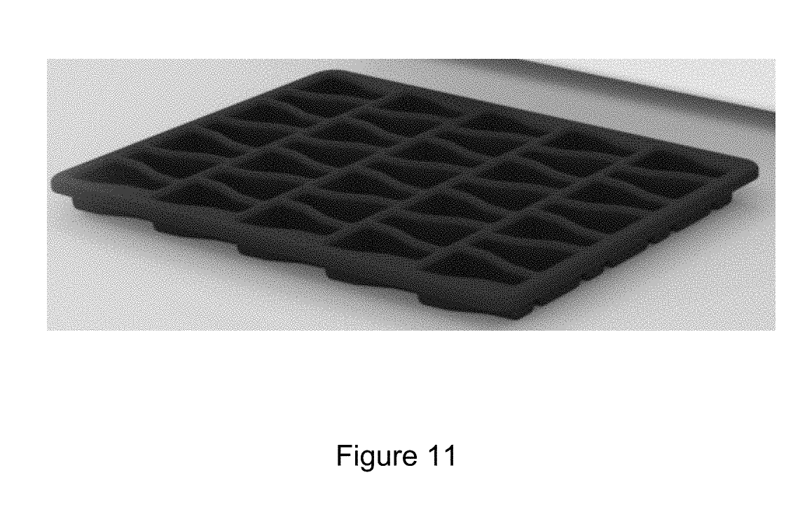

To maximise the number of recesses 304 on a support member 303 of a particular size (or alternatively to minimise the size of the support member required for a particular number of recesses), the recesses 304 can be tessellated, although this is not essential. The longitudinal axes of the recesses are preferably parallel to each other. The recesses 304 may be arranged in rows. For example, the support member 303 shown in FIGS. 9 and 10 has two rows, each row containing seven recesses. However, the support member 303 is not limited to this number of rows and recesses. The support member 303 may contain 1-15 rows, preferably 1-10 rows and more preferably 1-5 rows, such as 1, 2, 3, 4 or 5 rows. Each row preferably contains 1-10 recesses, more preferably 2-8 recesses and even more preferably 4-7 recesses, such as 4, 5, 6, or 7 recesses. FIG. 11 shows an embodiment with 5 rows and 7 recesses per row.

Within each row, adjacent recesses are conveniently orientated in opposite directions, such that adjacent recesses point to opposite sides of the support member 303. The recesses 304 are preferably arranged such that, within a row, the small end 304b of one recess is adjacent to the large end 304b of an adjacent recess. Such an arrangement is shown in FIGS. 9 and 10. When capped needle assemblies 200 are placed in the recesses 304, adjacent capped needle assemblies 200 preferably point in opposite directions. The recesses 304 may also be provided in other arrangements that result in a high number of number of recesses 304 on the support member 303, while keeping each recess 304 separate from the others.

The dimensions of the small end 304a and large end 304b of the recesses 304 and the axial length and the depth of the recesses 304 are preferably the same as those described for the recesses 104 of the first embodiment. The axial length of each recess 304 is preferably no more than twice the maximum width of the recess 304, or no more than 2.5 times the maximum width of the recess 304, or no more than three times the maximum width of the recess 304. The recesses 304 are also preferably bevelled rather than sharp-edged, as for the first embodiment.

The support member 303 can be any suitable material, such as metal or silicone, as in the first embodiment.

Although some of the above features have been described only in relation to the device 300 of the second embodiment, the person skilled in the art would appreciate that many of the features of the device 300 of the second embodiment can also be applied to the device 100 of the first embodiment. For example, the chamber 102 of the device 100 may also be made of stainless steel and optionally surrounded by an insulating material, such as a mineral wool. The outer surface of the device 100 may also be made from plastic, such as ABS plastic, and/or may have a ribbed structure. The device 100 may also have a lid similar to the lid 305 with chamber cover 350 of the second embodiment. The device 100 may have a display similar to the display 360 of the second embodiment in addition to or as an alternative to and LED indicator. The recesses 104 of the device 100 may be arranged on the support member 103 in a similar way to the recesses 304 of the second embodiment, e.g. tessellated. As already mentioned, the support member 103 of the device 100 is not limited to a disc shape, and could be other shapes such as a rectangle or any N-sided polygon, where N>3.

Features from the device 100 of the first embodiment may similar be used in the device 300 of the second embodiment. For example, the second embodiment could have a transparent lid or simple light displays without a timer.

For both embodiments, the number of recesses may vary depending on the size of the device. The support member 103, 303 has at least one recess, preferably 2 to 100 recesses, more preferably 2 to 50 recesses and even more preferably 4 to 35 recesses. In one example of the device, the support member comprises 30 to 40 recesses, preferably 35 recesses optionally arranged in five rows of seven recesses. Such an arrangement is shown in FIG. 11.

A device may typically have a length and width of approximately 200 to 300 mm, preferably 230 mm, and a height of approximately 50 to 70 mm, preferably 58 mm.

In a smaller example of the device 300, the supporting member may comprise 10 to 20 recesses, preferably 14 recesses optionally arranged in two rows of seven recesses.

Another device typically has a length and width of approximately 150 to 250 mm, preferably 190 mm, and a height of approximately 50 to 70 mm, preferably 58 mm.

For a portable sized device that can be carried around by the user, the support member 303 may comprise 2 to 8 recesses, preferably 3 to 5 recesses, such as 3, 4 or 5 recesses. In the case of 4 recesses, the recesses may be arranged in two rows of two recesses, or one row of four recesses.

The device 100, 300 is preferably powered by mains electricity, but may be powered by an optional battery, preferably a rechargeable battery, that provides power when the device is not connected to the mains. This is especially advantageous for a portable version of the device.

Although the support members 103 and 303 have been described as comprising recesses of one shape, the support member according to the invention may comprise recesses of different shapes. For example, there can be at least one recess having a first shape and at least one other recess having a second different shape.

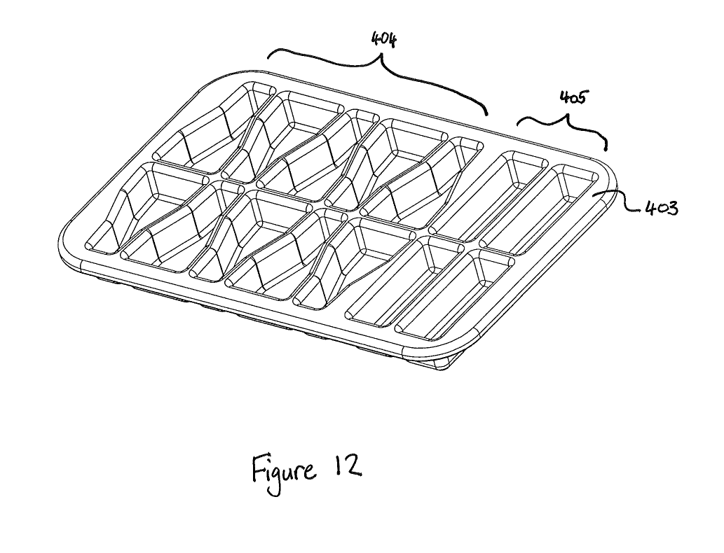

FIG. 12 shows a support member 403 according to the present invention that comprises a first set of recesses 403 having a first shape and a second set of recesses 404 having a second shape, wherein the first shape and the second shape are different.

The support member 403 is not limited to having two sets of recesses and may comprise a plurality of sets of recesses, wherein the recesses of each set have a different shape to the recesses of the other sets. For example, the support member may further comprise a third set of recesses having a third shape, wherein the third shape is different to the first shape and second shape.

In FIG. 12, the first set of recesses 404 has a first shape and dimensions that are identical to the above-described recesses 304. However, the first shape is not limited to the shape of recesses 304. For example, the first set of recesses 404 may have a first shape and dimensions that are identical to the above-described recesses 104.

Regardless of the first shape, the second shape is preferably different. Each recess of the second set 405 preferably has an elongated rectangular shape in plan view. Such a shape facilitates the reception and encapsulation of capped lancets, which typically have an elongated shape.

Preferably, each recess of the second set 405 has an axial length of at most 40 mm, preferably at most 38 mm, more preferably at most 35 mm. The axial length should be greater than 25 mm, preferably greater than 27 mm or preferably greater than 29 mm, most preferably greater than 32 mm. The most preferred length is 35 mm.

Preferably, each recess of the second set 405 will have a width of at most 25 mm, more preferably at most 20 mm, most preferably at most 15 mm. The most preferred width is 12 mm.

Each recess of the second set 405 preferably has a depth of at least 1.0 mm, more preferably at least 3.0 mm, most preferably at least 5.0 mm. The depth is preferably no more than 20 mm, more preferably no more than 15 mm, most preferably no more than 10 mm.

The length of each recess 405 is preferably more than twice its width, or 2.5 times its width, or more than three times its width.

Optionally, the depth of the recesses within the first set 404 is the same as the depth of the recesses of the second set 405 (and the depth of the other sets, in the case of more than two sets).

The recesses of the support member 403 are preferably arranged such that recesses of each set (i.e. recesses of the same shape) are physically grouped together on the support member 403. For example, the first set of recesses 404 may be arranged at one end of the support member 403 and the second set of recesses 405 may be arranged at the other end of the support member 403, as shown in FIG. 12.

The recesses within each set are optionally arranged in rows. The rows of one set preferably align with the rows of another set. In FIG. 12, the recesses within the first set are arranged in two rows and the recesses within the second set are arranged in two rows. The rows of the first set are aligned with the rows of the second set, so as to overall form two rows of recesses on the support member 403. The recess arrangements described for support members 103 and 303 may also be applied to support member 403.

The recess arrangement within one set may be different to the recess arrangement within the other sets. For example, the number of rows of recesses within the first set may be different to the number of rows of recesses within the second set, or the recesses of the first set may be arranged in rows, while the recesses of the second set may be arranged in a circular pattern, such as the arrangement of recesses shown in FIG. 4.

The recesses are not limited to the two shapes shown in FIG. 12. The recesses may have other shapes, depending on the shape of the sharps waste to be encapsulated. Each recess is preferably of a shape that generally follows the outline of a particular type of sharps waste to be encapsulated.

By providing recesses of different shapes on the same support member 403, the encapsulation of different types of sharps waste (e.g. pen needles, blood testing lancets and syringes) can be performed at the same time using the same device. Of course, different types of sharps waste could be received and encapsulated using recesses of just one shape. However, by providing recesses of different shapes, different types of sharps waste having different shapes can be received and encapsulated more effectively and efficiently.

As well as an apparatus, the invention also comprises a corresponding method of encapsulating a needle 201. The below method is described with reference to components of the device 100 of the first embodiment. However, the method is substantially the same when using the device 300 of the second embodiment. The method of the invention generally includes receiving into a recess 104 of a support member 103 a needle 201 contained in a corresponding plastic needle cap 206 and heating the support member 103 such that the plastic material of the needle cap 206 softens and flows to fully encapsulate the corresponding needle 201.

Preferably, plural needles 201 and corresponding plastic needle caps 206 are received into respective plural recesses 104 of the support member 103. This allows multiple pen needle assemblies 200 to be encapsulated at the same time.

The method preferably further comprises a step of cooling the support member 103 so that the softened plastic hardens whilst encapsulating the corresponding needle 201.

The method may further comprise removing the support member 103 and disposing of the encapsulated needle(s) 201.

The method may further comprise locking the lid 105 while heating and cooling.

The method may further comprise preventing the needle 201 contained in its corresponding plastic needle cap 206 from being received into a recess 104 with the needle 201 in the generally vertical direction.

The method may further comprise filtering air in the chamber 102 to remove toxic or unpleasant components.

The method may further comprise measuring the temperature of the air in the chamber 102 and unlocking the lid 105 when the temperature falls below a predetermined value. The predetermined value is preferably in the range 30° C. to 70° C., preferably 40° C. to 60° C., more preferably 40° C. to 50° C.

Preferably, the cooling comprises active cooling obtained by pumping air through the chamber 102.

Preferably, the support member 103 is heated to a temperature of from 180° C. to 330° C., preferably 180° C. to 280° C., more preferably 190° C. to 250° C., more preferably 200° C. to 240° C., more preferably 210° C. to 230° C., most preferably 220° C. or 230° C. Keeping the temperature at or under 240° C. helps to avoid the plastic material of the plastic cap 206 from burning and creating unpleasant fumes. Keeping the temperature above 180° C. helps to ensure that the plastic is softened to an extent that it is able to flow around the needle in a reasonably short period of time.

From the user's point of view, the method of operating the device 100 of the first embodiment of the present invention may comprise the following steps:
The lid opening button 118 is pressed to open the lid 105.
One or more pen needle assemblies 200 are placed in respective separate recesses 104 on the support member 103.
The lid 105 is manually closed by the user.
Sensors detect when the lid 105 is fully closed and an LED indicator provides an indication of whether the lid is open or not.
The user presses the start button 117.
The device 100 waits a period of time before starting the heating process, for example 30 seconds. Once the period of time has elapsed, the 105 lid locks and cannot be opened until finished. The period of time is designed to allow the user to cancel the procedure, for example to add additional pen needle assemblies 200.
The heating process is activated for a predetermined period of time, for example, 20 minutes to 40 minutes. The period of time is selected to ensure that the plastic softens and flows to the extent required to encapsulate the needle 201.
An LED indicator indicates when heating is taking place.
Once heating is finished, a cooling process is activated which may optionally include the activation of a pump 111 to draw air through the chamber 102, thereby speeding cooling. Fumes from the chamber 102 are filtered. An LED indicator, preferably a blue LED indicator, indicates the cooling process.
Once the temperature in the chamber 102 is below a predetermined value, e.g. 50° C., the lid unlocks and an LED indicator is lit or changes colour to indicate the process as finished.
The user then opens the lid 105 by pressing the lid opening button 118.
The support member 103 may be removed by the user, using the optional hole 120, and the encapsulated needle briquettes may be disposed of in the bin.

From the user's point of view, the method of operating the device 300 of the second embodiment of the present invention may comprise the following steps:
The lid opening button 318 is pressed to open the lid 305.
One or more pen needle assemblies 200 are placed in respective separate recesses 304 on the support member 303.
The lid 305 is manually closed by the user.
Sensors detect when the lid 305 is fully closed and a message on the display 360 provides an indication of whether the lid is open or not.
The user presses the start button 317.
The device 100 waits a period of time before starting the heating process, for example 30 seconds. Once the period of time has elapsed, the 305 lid locks and cannot be opened until finished. The period of time is designed to allow the user to cancel the procedure, for example to add additional pen needle assemblies 200.
The heating process is activated for a predetermined period of time, for example, 20 minutes to 40 minutes. The period of time is selected to ensure that the plastic softens and flows to the extent required to encapsulate the needle 201.
The display 360 displays a countdown of the time remaining when heating is taking place.
Once heating is finished, a cooling process is activated which may optionally include the activation of a pump 311 to draw air through the chamber 302, thereby speeding cooling. Fumes from the chamber 302 are filtered. A message or a countdown on the display 360 indicates when this cooling process is taking place.
Once the temperature in the chamber 302 is below a predetermined value, e.g. 40° C., the lid unlocks and a message on the display 360 indicates the process as finished.
The user then opens the lid 305 by pressing the lid opening button 318.
The support member 303 may be removed by the user and the encapsulated needle briquettes may be disposed of in the bin.

When the support member 303 is made of silicone, this facilitates removal of the encapsulated needles by bending of the silicone so that the briquettes can be popped into a bin without touching them.

1. A device for encapsulating a needle contained in a corresponding plastic needle cap, the device comprising:
a support member having at least one recess, each said at least one recess being configured to receive a needle contained in a corresponding plastic needle cap; and
a heating element configured to heat the support member such that the plastic material of the needle cap softens and flows to encapsulate the corresponding needle;
wherein each said at least one recess has dimensions such that, after heating, the needle is fully encapsulated by the plastic material of the corresponding needle cap.

2. The device according to claim 1, wherein the support member is a tray.

3. The device according to claim 1, wherein the support member has a plurality of recesses.

4. (canceled)

5. The device according to claim 3, wherein the recesses are tessellated.

6-7. (cancelled)

8. The device according to claim 1, wherein each said at least one recess is configured to receive only a single needle and corresponding plastic needle cap.

9. The device according to claim 1, wherein each said at least one recess is shaped to receive a needle and corresponding plastic needle cap substantially horizontally.

10. The device according to claim 1, wherein each said at least one recess has a length of at most 45 mm, a width of at most 30 mm and a depth of at most 20 mm.

11. The device according to claim 1, further comprising:
a chamber containing the support member and the heating element; and
an openable lid configured to seal the chamber when the lid is in the closed position.

12. (canceled)

13. The device according to claim 11, further comprising:
an inlet configured to allow air into the chamber; and
an outlet configured to allow gases inside the chamber to leave the chamber;
wherein the inlet includes a one-way valve configured to prevent gases leaving the chamber via the inlet and
wherein the outlet includes a filter.

14-15. (canceled)

16. The device according to claim 11, further comprising:
a temperature sensor for measuring the temperature in the chamber; and
a locking mechanism;
wherein the locking mechanism is configured to prevent the lid from being opened by a user when the measured temperature in the chamber is above a predetermined value.

17. (canceled)

18. The device according to claim 13, further comprising a pump configured to pump air through the chamber, wherein the pump is configured to activate after the heating element has been deactivated, and wherein the pump is configured to deactivate after the measured temperature in the chamber falls below a predetermined value.

19-21. (canceled)

22. The device according to claim 1, wherein the heating element is configured to heat the support member to a temperature of from 180° C. to 330° C.

23. (canceled)

24. The device according to claim 11, wherein the lid is transparent.

25. The device according to claim 1, wherein the support member is removable from the chamber.

26-27. (canceled)

28. The device according to claim 1, wherein the support member is made from silicone or stainless steel.

29-31. (canceled)

32. A method of encapsulating a needle contained in a corresponding plastic needle cap, the method comprising:
directly receiving into a recess of a support member a needle contained in a corresponding plastic needle cap; and
heating the support member such that the plastic material of the needle cap softens and flows to fully encapsulate the corresponding needle.

33. The method of claim 32, wherein plural needles and corresponding plastic needle caps are received into respective plural recesses of the support member.

34. The method of claim 32, further comprising a step of cooling the support member so that the softened plastic hardens whilst encapsulating the corresponding needle, wherein the cooling comprises active cooling created by pumping air through the chamber.

35-37. (canceled)

38. The method of claim 32, further comprising preventing the needle contained in its corresponding plastic needle cap from being received into a recess with the needle in the generally vertical direction.

39-42. (canceled)

43. A support member for a needle encapsulating device, said support member comprising one or more recesses for supporting a capped needle assembly.
